(19) United States
(12) Patent Application Publication
(10) Pub. No.: US 2024/0293115 A1
(43) Pub. Date: Sep. 5, 2024

(54) SUTURE DELIVERY DEVICE AND METHODS OF USE THEREOF

(71) Applicant: xDot Medical Inc., Maple Grove, MN (US)

(72) Inventors: Aashiish Agnihotri, Maple Grove, MN (US); Jeffrey Sandstrom, Scandia, MN (US); Steven M. Maurer, Minneapolis, MN (US); Trevor Laughlin, Minneapolis, MN (US)

(21) Appl. No.: 18/662,909

(22) Filed: May 13, 2024

Related U.S. Application Data
(63) Continuation-in-part of application No. 18/508,193, filed on Nov. 13, 2023.
(60) Provisional application No. 63/424,747, filed on Nov. 11, 2022.
(60) Provisional application No. 63/642,687, filed on May 4, 2024.

(51) Int. Cl. A61B 17/04; A61B 17/00

(52) U.S. Cl. CPC A61B 17/0469; A61B 2017/00663; A61B 2017/0409; A61B 2017/0464; A61B 2217/005

(57) ABSTRACT

A delivery device for suture deployment for post-procedure closure of an access site after removal of an interventional device is disclosed. The delivery device includes a distal assembly comprising an elongate member, a proximal housing comprising one or more control elements for controlling one or more components of the delivery device, and a plurality of delivery tubes extending distally from the proximal housing into the elongate member. Each of the plurality of delivery tubes includes a suture anchor stored within a distal portion of that delivery tube, and a push rod configured to push the suture anchor distally out of the distal portion of that delivery tube for deployment of the suture anchor within a target tissue.

### CLAIM OF PRIORITY

This application claims priority to and is a continuation-in-part of U.S. application Ser. No. 18/508,193 filed on Nov. 13, 2023 which in turn claims priority to U.S. Provisional Appl. No. 63/424,747 filed on Nov. 11, 2022. This application also priority to U.S. Provisional Appl. No. 63/642,687 filed on May 4, 2024. The disclosure of each of these priority applications is incorporated herein by reference in its entirety.

### FIELD OF THE DISCLOSURE

The present disclosure generally relates devices and methods for gaining percutaneous access to the lumen of a blood vessel and for subsequently closing the access site into the blood vessel.

### BACKGROUND OF THE DISCLOSURE

Health practitioners frequently use sutures to close various openings such as cuts, punctures, wounds, incisions, or otherwise joining tissue portions in various places in the human body. Generally, sutures are convenient to use and function properly to hold openings in biological tissue closed, thereby aiding in blood clotting, healing, and prevention of scarring.

For example, in many interventional procedures, an interventional device is introduced through the patient's artery or vein (percutaneous approach). For example, in the treatment of vascular disease, such as arteriosclerosis, it is a common practice to invade the artery and insert an instrument (e.g., a balloon or other type of catheter) to carry out a procedure within the artery. Such procedures usually involve the percutaneous puncture of the artery so that an insertion sheath can be placed in the artery and thereafter instruments (e.g., a catheter) can pass through the sheath and to an operative position within the vascular system. While these procedures present various medical advantages, the potential for bleeding during the procedure can present dangers to the patient. Moreover, after the particular interventional procedure has been performed, the access hole in the vessel wall must be closed. A number of prior vascular closure devices and methods have been developed in attempting to provide a solution for the problem of closing a hole in the vessel wall.

In some cases, medical suturing systems are utilized to “pre-close” the access site or puncture in the vessel wall by positioning one or more stitches adjacent to the interventional device that result in hemostasis of the vessel wall around the interventional device during the procedure. In some other cases, sutures need to be placed around an access site after a procedure is performed (i.e., an interventional device is inserted without placement of sutures) to “post-close” the access site. After the procedure is completed and the interventional device(s) are removed, the stitches positioned by the medical suturing system (before or after insertion of an interventional device within an access site) are utilized to permanently or reversibly (e.g., for reinsertion of the interventional device) close or cinch the access site. Often the interventional procedures are performed via minimally invasive approaches and/or percutaneously where direct visualization is not possible. Use of traditional sutures and suturing systems becomes challenging for deployment of sutures for such procedures because direct visualization is not possible.

The current disclosure describes devices and methods directed towards solving some of the issues discussed above.

### SUMMARY OF THE DISCLOSURE

Disclosed scenarios provide a delivery device for suture deployment. The delivery device may include a distal assembly comprising an elongate member and a dilator having a diameter of about 10-12 French. The delivery device also includes a proximal housing comprising one or more control elements for controlling one or more components of the delivery device, and a plurality of delivery tubes extending distally from the proximal housing into the elongate member. Each of the plurality of delivery tubes may include a suture anchor stored within a distal portion of that delivery tube, and a push rod configured to push the suture anchor distally out of the distal portion of that delivery tube for deployment of the suture anchor within a target tissue. A sheath may be removably mounted over at least a distal portion of the elongate member.

Optionally, a guidewire lumen may extend between a distal end and a proximal end of the delivery device.

In various implementations, the delivery device may include a blood return channel extending between a distal port disposed within the distal assembly and a proximal port disposed within the proximal housing. The blood return channel may be configured to provide an indication of proper positioning of the distal assembly within a target tissue based on presence of blood in the proximal port. Optionally, the blood return channel may be a passageway defined by an inner guidewire lumen and an outer tube. Optionally, a length of the dilator may be configured to plug the access site to prevent leakage of blood from the access site such that presence of blood in the proximal port provides the indication of proper positioning of the distal assembly within the target tissue. The length of the dilator may be about 1-5 inches.

In certain implementations, the distal assembly may include a stabilizer. In various embodiments, the stabilizer may have a total wing span of about 2 mm to about 6 mm in an expanded state Optionally, the stabilizer may be disposed between the dilator and the elongate member and/or may have a diameter that is substantially similar to that of the dilator in an undeployed state. Additionally and/or alternatively, distal movement of an outer tube within the elongate member may cause deployment of the stabilizer for providing temporary fixation of the delivery device at a location in the target tissue that will correctly position the suture anchors upon deployment.

In various implementations, the elongate member may include a proximal tubular portion and a distal conical portion. The distal conical portion may include a plurality of guiding features configured to position the plurality of delivery tubes in a desired configuration upon deployment.

A passageway may extend from a distal end of the elongate member into the proximal housing. The passageway may be configured to receive a guidewire lumen, the plurality of delivery tubes, and an outer tube configured to define a blood return channel. Each delivery tube may include an elongate tubular member extending distally from a delivery tube holder in the proximal housing, into the passageway. A push rod may extend distally into a lumen of each delivery tube from a push rod holder in the proximal housing. Optionally, the proximal housing may include a rack and pinion control mechanism configured to be actuated by a flip handle such that rotational motion of the flip handle causes distal advancement of the delivery tube holder and the push rod holder. Additionally and/or alternatively, the delivery device may include a resilient member configured to stop distal movement of the delivery tube holder when in a fully compressed state. As such, distal movement of the push rod holder upon stopping of distal movement of the delivery tube holder causes the push rod to deploy a suture anchor by pushing it out the distal end of the delivery tube within the target tissue. In some embodiments, each of the plurality of delivery tubes comprises a distal portion that is axially offset or flares outwardly with respect to the proximal portion of that delivery tube such that the sutures are deployed to form an area having a rectangular cross section of about 3 mm×8 mm.

Optionally, each of the plurality of delivery tubes may include a slot for receiving a distal end of a suture, the distal end of the suture being couple with the suture anchor. A positioning element may be disposed within the passageway to cause flaring of the plurality of delivery tubes outwardly with respect to the passageway upon deployment.

In various scenarios, a method of deploying sutures using the above delivery device may include advancing a distal end of the delivery device into a previously enlarged access site until the access site is plugged to prevent leakage of blood from the access site, deploying a stabilizer upon observation of blood return, distally advancing the plurality of delivery tubes into the target tissue, and distally advancing each push rod into a lumen of that delivery tube to cause deployment of that suture anchor upon detecting that the plurality of delivery tubes have reached a maximum advancement position.

In certain other scenarios, a suture delivery device for suture deployment is disclosed. The delivery device may include an outer tube disposed concentrically around a guidewire lumen to form a blood return channel outside the guidewire lumen and a passageway defined by the delivery device. The passageway is configured to house the outer tube and a plurality of delivery tubes. The plurality of delivery tubes may extend distally from a delivery tube holder. Each delivery tube may include a suture anchor stored within a distal portion of that delivery tube, and a push rod extending from a push rod and configured to push the suture anchor distally out of the distal portion of that delivery tube for deployment of the suture anchor within a target tissue. The delivery device may also include a resilient member configured to stop distal movement of the delivery tube holder when in a fully compressed state. Distal movement of the push rod holder upon stopping of distal movement of the delivery tube holder may cause, via the push rod, deployment of suture anchor within the target tissue.

In various implementations, the delivery device may include a control mechanism configured to cause distal advancement of the delivery tube holder and the push rod holder.

### BRIEF DESCRIPTION OF THE DRAWINGS

FIG. 1A is a perspective view of an example delivery device.

FIG. 1B is a top view of the delivery device of FIG. 1A.

FIG. 1C is a bottom view of the delivery device of FIG. 1A.

FIG. 1D is an exploded of the delivery device of FIG. 1A along a longitudinal axis of the delivery device.

FIG. 1E is a cross sectional view of the delivery device of FIG. 1A along the XY plane.

FIG. 2A is a schematic diagram illustrating deployment of an example stabilizer.

FIG. 2B is a schematic diagram illustrating deployment of another example stabilizer

FIG. 3A is an enlarged view of an elongate member of the delivery device of FIG. 1A and

FIG. 3B is a cross section view of the elongate member along the YZ plane.

FIG. 4A is an enlarged front perspective view of a conical portion of the elongate member of FIG. 3A.

FIG. 4B illustrates a cutaway view of conical portion with the delivery tubes.

FIG. 4C illustrates a cutaway view of conical portion without the delivery tubes.

FIG. 4D illustrates the delivery tubes without the conical portion.

FIG. 5 illustrates an example positioning element.

FIGS. 6A-6E illustrate various types of suture anchors.

FIG. 7 is a schematic illustration of unequal length delivery tubes deployed within a target tissue.

FIGS. 8A-8C illustrate cross section views of the housing of the device of FIG. 1A in a neutral position, in a deployment position, and in a retracted position, respectively.

FIG. 9 illustrates suture anchors stored within the distal ends of delivery tubes.

FIG. 10 is a flowchart illustrating an example method of using a delivery device of the current disclosure for deployment of sutures.

FIG. 11A is a schematic illustration of a portion of the example method of FIG. 9.

FIG. 11B is a schematic illustration of a portion of the example method of FIG. 9.

FIG. 11C is a schematic illustration of a portion of the example method of FIG. 9.

FIG. 11D is a schematic illustration of a portion of the example method of FIG. 9.

FIG. 12A is a perspective view of a distal portion of an example delivery device for post closure of an access site.

FIG. 12B is a perspective view of a distal portion of the example delivery device illustrating the stabilizer in an expanded state.

FIG. 12C is a perspective view of a distal portion of the example delivery device illustrating the delivery tubes being advanced out of the conical portion.

FIG. 13 illustrates a top view of the locations of the deployed sutures.

### BRIEF DESCRIPTION OF DISCLOSED EMBODIMENTS

The devices and methods of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, proximal, distal, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

The terms “proximal” and “distal” are used herein with reference to an operator manipulating the delivery device. As used herein, the term “proximal” means closest to the operator (less into the body) and “distal” means furthest from the operator (further into the body).

The term “suture” as used herein can be monofilament or multifilament elongate flexible tensioning devices made of flexible or non-flexible material with sufficient tensile strength for providing a desired force. The suture can be a strand, a wire, a cord, a fiber, a yarn, a filament, a cable, a thread, or the like, and these terms may be used interchangeably. Suture materials may generally be broken into two categories, absorbable (capable of being broken down and absorbed into the body) and non-absorbable (should be manually removed from the body). Example materials include but are not limited to polyglycolic acid, polylactic acid (PLA), polypropylene, polyester, silicone, polyurethane, stainless steel, nickel titanium alloy, nylon, Kevlar fiber, or others.

As used herein, the terms “closure” “closed”, “closing” refer to reducing the size (e.g., diameter, area, volume, etc.) of an opening in a biological structure (e.g., a blood vessel) from an initial size to a smaller size. The opening may be an aperture or a passageway that natural or surgical created. An “access site” refers to a surgically created opening created in a biological structure for, for example, an interventional procedure.

Methods and systems for deploying sutures for closure of an opening (e.g., by cinching) are disclosed herein. While the examples provided in this disclosure generally relate to suture delivery devices (or “delivery devices”) for percutaneous management of vascular access sites (i.e., in blood vessels), disclosed systems and methods may be used for delivery or deployment of sutures to other access sites or openings, such as, but not limited to, access sites or openings in various internal organs. For example, disclosed embodiments may be used for management of access sites or openings in the heart (e.g., inside or outside of the heart) or the gastrointestinal tract. For example, the suture delivery devices of the current disclosure can be used to deploy sutures that can be used to treat an anatomical valve, such as a heart valve, including heart valves that may be weakened or stretched, or have other structural defects, such as congenital defects, that cause them to close improperly. The suturing system includes a suture delivery device for deployment of sutures that can be used to close or reduce a variety of other tissue openings, lumens, hollow organs or natural or surgically created passageways in the body. Particularly, a delivery system for delivery or deployment of sutures for closure of various openings are described.

The delivery system of the current disclosure may be used to deploy sutures for closing openings (e.g., by cinching when sutures are tensioned) using closure systems and devices described in, for example, U.S. Pat. No. 11,382,609, U.S. Patent Application Publication No. 20220175356, U.S. Patent Application Publication No. 20230149005, and U.S. Patent Application Publication No. 20230309979, the disclosures of which are incorporated herein by reference in their entirety. The delivery system of the current disclosure may be used with any now or hereafter known suture systems and closure devices.

In some embodiments, the present disclosure relates to methods and devices for deploying sutures for “pre-closing” an opening (e.g., in a blood vessel, in a heart valve, etc.). For example, the methods and devices may be used deploying sutures for closure of access sites into blood vessels wherein the sutures are applied before the vessel is accessed with an interventional device such as a sheath, catheter, or cannula.

In some instances, a micro-puncture technique is used whereby the vessel is initially accessed by a small gauge needle, and successively dilated up to the size of the access device. For percutaneous access, a puncture is made from the skin, through the subcutaneous tissue layers to the vessel, and into the vessel itself. In certain types of procedures, it is advantageous to “pre-close” the access site, for example if the access site is significant in size, if the access site is difficult to access, or if there is a heightened risk of inadvertent sheath removal. The term “suture pre-close” refers to deploying sutures before the vessel is accessed with the interventional device. The ability to gain rapid hemostatic control of the access site can be critical. In an open surgical procedure, a suture is sometimes placed into the vessel wall in a U-stitch, Z-stitch, or purse-string pattern prior to vessel access. The access site is made through the center of this stitch pattern. The suture may be tensioned around the interventional device during the procedure, or the suture may be left loose. If the interventional device is inadvertently removed from the access site, rapid hemostasis may be achieved by applying tension to the ends of the suture. After removal of the interventional device from the arteriotomy, the suture may then be appropriately manipulated to achieve reversible and/or permanent homeostasis.

In percutaneous procedures, it is not possible to insert a suture in the manner described above. In these procedures, if suture pre-close is desired, a percutaneous suture-based vessel closure device would need to be used. However, current percutaneous suture-based vessel closure devices require previous dilatation (widening) of the initial needle puncture or access site to be inserted into the vessel, and are designed to be placed after the interventional device has been inserted into, and in some cases removed from the access site. In this manner, the dilatation has been accomplished by the interventional device and dilator itself. In view of this, current suture-based vessel closure devices have certain limitations for use in pre-closure of an access site. To accomplish pre-closure with these devices, a dilator or dilator/sheath combination needs to be initially inserted into the vessel over a guidewire to dilate the initial puncture, and then exchanged for the closure device, with the difficulty of maintaining hemostasis during this exchange.

Another limitation is that once the suture is placed in the vessel with the suture-based vessel closure devices, it is likewise difficult to maintain hemostasis during removal of the suture-based vessel closure device and insertion of the interventional device. Similarly, once the interventional device is removed, it is difficult to maintain hemostasis before the final suture knot is tied. Or, if the suture is pre tied, it is difficult to maintain hemostasis before knot is pushed into place. In addition, current suture-based vessel closure devices do not have any means to gain rapid access to the suture ends to apply tension in the instance of inadvertent interventional device removal.

Certain procedures, for example intervention of the carotid arteries, offer additional clinical challenges. In a transcervical approach to treatment of the internal carotid artery and/or the carotid artery bifurcation, the distance from the access site to the treatment site is usually less than 5-7 cm. In the case of the Abbott PROSTAR or PERCLOSE suture-based closure devices, the vessel entry device requires about a 15 cm length into the vessel in order to provide the ability to pull out a guidewire. With other devices, there are no methods or features for limiting or controlling the amount of egress of these device components in the vessel. Therefore it is desirable to limit the length of the suture delivery device or any associated accessories (needle puncture, guidewire, micro introducer, dilator, or sheath itself), to remove risk of incursion into the plaque zone and reduce the risk of generating embolic particles.

The devices and methods disclosed herein aim to improve upon at least one of the aforementioned problems. However, it shall be understood that the disclosure herein is not limited to merely solving these specific problems. Additionally, while the devices and techniques disclosed herein are described with respect to a human body or patient, it is understood that the devices and techniques may in suitable circumstances be applied to a non-human patient (i.e., in veterinary medicine).

In various implementations, the current disclosure describes a suture delivery device that can perform the dilation of an initial access site (e.g., a needle puncture), and therefore does not require previous dilation by a separate device or by a procedural sheath dilator. The suture delivery device can place, anchor, or otherwise deploy one or more sutures in proximity of an access site for subsequent cinching (around an interventional device or otherwise) of the access site using a closure device to provide hemostasis to the access site. Although the devices are discussed in connection with promoting hemostasis, the devices and methods disclosed herein can be used in other applications to achieve different results, for example to provide management of access sites to the heart or gastrointestinal tract. Similarly, the disclosed suture delivery device can place, anchor, or otherwise deploy one or more sutures in proximity of any opening for subsequent cinching of the opening using a closure device. For example, the anchors and sutures of the current access and closure device may be deployed proximate to and/or around an opening in the heart.

The disclosed suture device limits the length of the dilator of the suture delivery device inserted within the vessel (or any associated accessories) to about 0.5 inches to about 1.5 inches, about 0.75 inches to about 1.25 inches. Optionally, during deployment of the sutures, the sutures are slack (i.e., do not include tension) in order to avoid severing of the sutures by the interventional device during insertion. Sutures are tensioned after insertion of the interventional device.

The devices of this disclosure can, therefore, decrease resources (e.g., personnel time, materials, etc.) used to control or substantially eliminate bleeding/oozing/exuding fluids. The device can be applied by a wide range of individuals to quickly stem leakage and/or promote wound healing. Disclosed aspects may be employed percutaneously (i.e., without direct visualization of the user). However, it is understood that the devices and methods described herein can also be used under direct visualization by the user or with indirect visualization using, for example, a surgical endoscope.

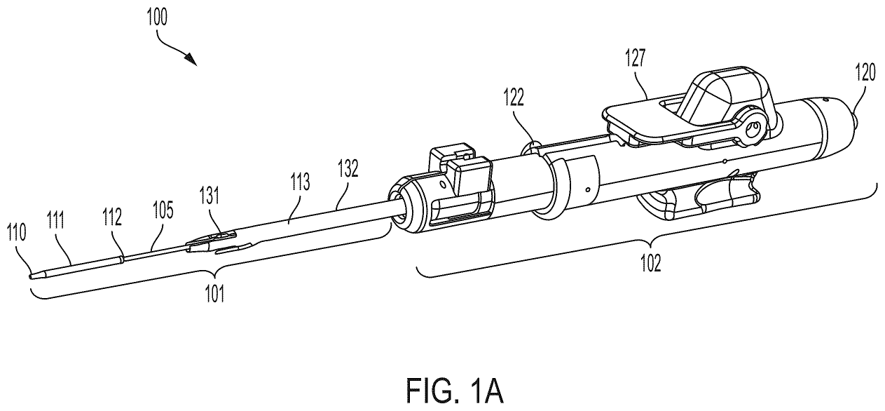

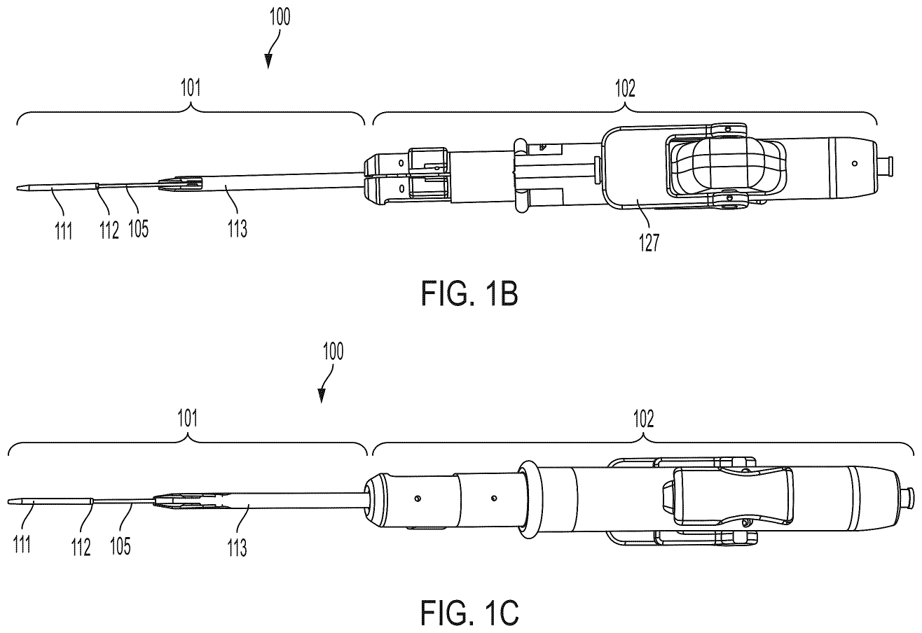

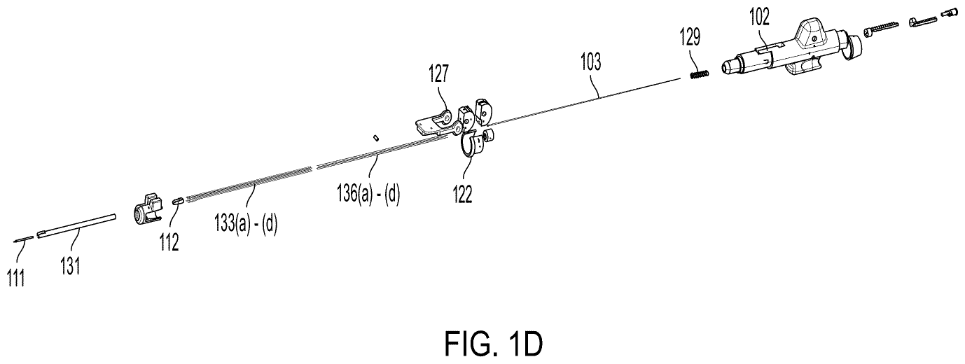

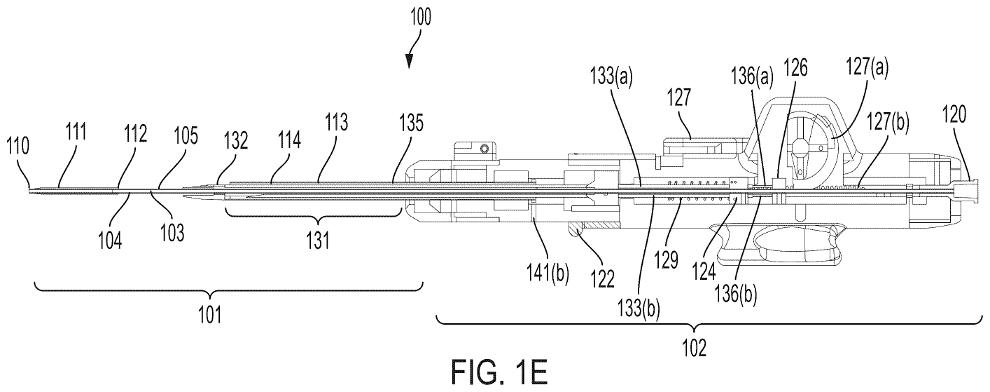

Referring now to the drawings, and in particular to FIGS. 1A-1E, an example suture delivery device 100 is shown and described.

The suture delivery device 100 generally includes a distal assembly 101 and a proximal housing 102 having control elements or actuators (e.g., such as a movable actuation lever 127 and/or actuation button 122) and control mechanisms configured to control one or more components of the delivery device. The type, number, and shape of the control elements can vary. The movable actuation lever 127 is a flip handle controls movement of suture delivery tubes and pusher rods for deployment of sutures via suture anchors (as discussed below). The actuation button 122 controls deployment of a stabilizer as discussed below. Optionally, the housing 102 may include one or more interfacing elements (not shown here) such as clips, tabs, slots, or the like disposed on the body of the housing for removable attachment of closure devices (e.g., tensioning devices) or components thereof.

A continuous guidewire lumen 103 is provided inside the device 100 between the distal tip 110 and the proximal tip 120 for tracking the suture delivery device 100 over a guidewire (not shown). Optionally, the guidewire lumen may extend between the proximal tip 120 and a proximal end of the dilator 111 such that the dilator lumen is contiguous with the guidewire lumen. The distal exit of the guidewire lumen 103 provides a smooth transition to the guidewire, so the device can smoothly be inserted into an opening over the guidewire. Thus, the diameter of the guidewire lumen 103 may be close to the diameter of the guidewire itself when it exits the distal tip 110. For example, for compatibility with an 0.035″ or 0.038″ guidewire, the distal tip of the device can have a guidewire lumen of from 0.039″ to 0.041″ as it exits the tip (although it could be slightly larger for the remainder of the device). While the current disclosure describes that the guide wire lumen may extend along the entire length of the delivery device, such that a guidewire can ride along the entire length of the delivery device and exit out the proximal end, it is not so limiting. The guidewire may exit at a point in the delivery device that is distal to the proximal tip 120. As discussed below, the suture delivery device allows for deployment of the sutures without the need to remove the guidewire.

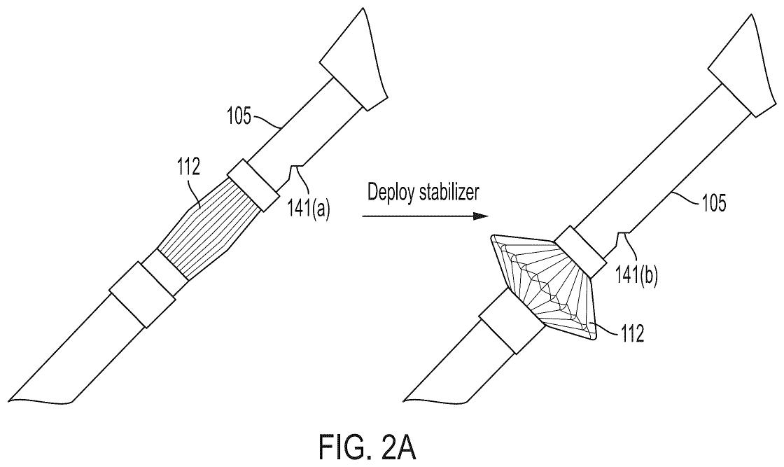

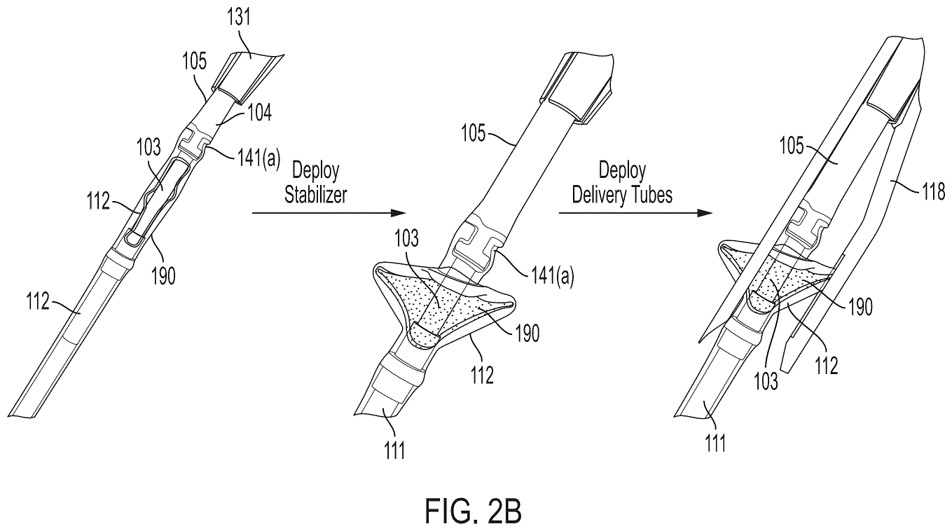

A blood return channel 104 is provided between a distal port 141(a) (shown in FIGS. 2A and 2B) within the distal assembly and a proximal port 141 (b) within the proximal housing (shown in FIG. 1E). The distal port 141 (a) of the blood return channel 104 is positioned relative to and/or in proximity of the stabilizer 112 such that when the stabilizer 112 is properly positioned within a vessel (e.g., completely within the vessel), blood pressure causes blood to flow proximally into the distal port 141 (a), through the blood return channel 104, and to the proximal port 141 (b) in the proximal housing 102. Presence of blood in the proximal port 141 (b) provides an indication that the stabilizer 112 has entered the blood vessel and may be actuated to the “open” position (as discussed below). The proximal port 141 (b) may comprise a blood exit port, a clear tube or receptacle in which blood is visible, or the like. It should be understood that a wide variety of alternative sensors might be used, including electrical pressure sensors, electrolytic fluid detectors, or the like for determining the correct positioning of the stabilizer within a vessel. In some embodiments, the blood return channel 104 may be a passageway that is concentrically provided around at least a portion of the guidewire lumen such that blood flows in a space outside the guidewire lumen wall and inside an outer tube 105 disposed concentrically around the guidewire lumen. For example, FIG. 2B shows the outer tube 105 disposed around the guidewire lumen 103 to define a blood return channel 104. Optionally, a separate tube may be provided for the blood return channel.

In various embodiments, the distal assembly 101 comprises a dilator 111, a stabilizer 112, and an elongate member 113. A removable sheath 114 is mounted over the elongate member 113 and configured to cover at least a proximal portion of the elongate member 113. In various embodiments, the sheath 114 may be configured to cover the delivery tubes before deployment in order to prevent clogging the delivery tubes (e.g., with tissue, blood clots, etc.) as the delivery device is advanced within an opening to deploy the sutures. The sheath may be removed before deployment of the sutures by manual peeling away and/or by retraction (manually or mechanically). The sheath may be made from any suitable material such as, without limitation, polytetrafluoroethylene (PTFE), silicone, rubber, polyurethane, polyethylene, or the like, and may closely conform to the shape and size of the elongate member 113.

In various embodiments, the dilator 111 is made from a soft material and is configured to enlarge or dilate an initial puncture (e.g., made using a needle) made by a surgeon to access a vessel or other opening where sutures need to be deployed. The dilator is configured to allow for easy insertion of the delivery device through soft tissue and/or across the target tissue such as the vessel wall or septal wall. In this regard, the dilator has features (e.g., include size, shape, materials, and/or material properties) that are particularly adapted for dilating a particular opening. For example, when the dilator 111 is configured to dilate the initial puncture in a vessel as the delivery device enters the vessel, the dilator is constructed from materials and dimensioned to enlarge an initial puncture made within a vessel. In various embodiments, the length of the dilator is about 0.5 inches to about 1.5 inches, about 0.75 inches to about 1.25 inches, about 0.5 inches, about 0.75 inches, about 1 inch, about 1.25 inches, about 1.5 inches, about 2 inches, or the like. As discussed above, the length of the dilator is significantly less than those of existing suture delivery devices because the delivery device of the current disclosure does not require removal of a guidewire during deployment of the sutures. As such, the dilator does not need to have a length that is configured to provide support (once the guidewire has been removed) to the existing delivery devices that require removal of guidewire during deployment of the sutures.

In various embodiments, the dilator 111 is made from a soft material such as, for example, polyurethanes, polyesters, polymeric family/class such as polyamides including nylons and polyether block amides (PEBAX-72D), or the like. The soft material allows for bending of the dilator upon deployment within a vessel. In addition, the dilator may have a tapered distal portion so there are no abrupt transitions as the dilator enters an opening over a guidewire. For example, the leading edge of the distal dilator tip (i.e., the distal tip 110) may be radiused, for example 0.050″ to 0.075″ radius for compatibility with an example 0.035″ or 0.038″ guidewire and may gradually taper into a slightly larger radius of about 0.070 to about 0.1″ (i.e., about 5 to 8 French).

The stabilizer 112 is provided between and interfaces with (e.g., via friction fit or other suitable connection) a proximal end of the dilator 111 and a distal end of the outer tube 105, and provides a positioning reference for deployment of the sutures. In an undeployed or collapsed state, the stabilizer 112 is configured to have a diameter that is substantially similar to (or slightly smaller) that of the dilator 111. In some embodiments, the distal end of the stabilizer is at least partially inserted within the proximal end of the dilator 111. As discussed above and shown in FIGS. 2A and 2B, the distal port 141 (a) of the blood return channel 104 is positioned relative to and/or in proximity of the stabilizer 112, such that blood return is indicative of current positioning of the stabilizer 112. Specifically, the distal port may be located within a threshold distance of the proximal end of the stabilizer such that blood return is indicative of the entire length of the stabilizer 112 being across the target tissue within which the sutures are to be deployed (e.g., a vessel wall or a heart septum wall). The stabilizer 112 may be deployed by expansion of the stabilizer diameter upon observation of blood return such that in a deployed or expanded state the stabilizer rests against the target tissue providing temporary fixation of the delivery device at a location that will correctly position the sutures upon deployment. Expansion of the stabilizer close to or against the target tissue prevent unnecessary movement and/or dragging of the stabilizer within the vessel (or organ) thereby reducing the risk of damage to the walls of the vessel and also allows for the dilator length to be minimized.

In an example embodiment, shown in FIG. 2A, the stabilizer comprises a cylindrical mesh made from a suitable material (e.g., Nitinol, stainless steel, plastic or any combination thereof) and that flares outwards (i.e., transversely with respect to a central axis of the delivery device) upon deployment to create an approximate disc shape having a longitudinal length that is less than that of the longitudinal length of the cylindrical mesh in a collapsed state. Optionally, such a configuration of the stabilizer may also prevent entrapment of debris or particles (e.g., tissue, blood clots, etc.) within the stabilizer as it moves from a collapsed state to an expanded state (or vice versa)—in addition to the anchoring and accurate positioning functions.

FIG. 2B shows another example stabilizer comprising two arms having a substantially semi circular profile that are disposed around the guidewire lumen such that it forms a cylindrical profile in a collapsed state. The arms may flare outwards (i.e., transversely with respect to a central axis of the delivery device) upon deployment to create an approximate disc shape (or saucer shape, diamond shape, or the like) having a longitudinal length that is less than that of the longitudinal length of the cylindrical mesh in a collapsed state.

Optionally, the stabilizer may be coated with and/or encased in a suitable polymer material such as Chronoprene, Silicone, PUR, PET, or the like to provide additional protection to the vessel wall or other target tissue. Additionally, the coating may be selected to prevent blood from entering the blood return channel through the stabilizer and prevent a false signal. For example, as shown in FIG. 2B, the stabilizer 112 is encased within the polymer 190 to prevent blood flow into the blood return channel from between the arms of the stabilizer.

Without limiting the disclosure, the stabilizer may be any now or hereafter known symmetrical or asymmetrical stabilizer such as, without limitation, a balloon that can be expanded from a collapsed state upon deployment, a stent, a disc, a sheath, a shape memory (e.g., Nitinol) mesh that can transform into a disc in a deployed state, polymer arms that are configured to fold symmetrically and/or asymmetrically either along a longitudinal axis of the delivery device (in a collapsed state) or transversely (upon deployment), shape memory (e.g., Nitinol) arms that are configured to be in a cylindrical shape (in a collapsed state) or fold reversibly upon deployment to create a pattern (e.g., an “X” shape) to create an anchor against the target tissue, or the like.

In various embodiments, the stabilizer 112 may be deployed by movement of the outer tube 105 distally such that the distal end of the elongate member pushes against the proximal end of stabilizer 112 to shorten or compress the axial length (longitudinal) of the stabilizer and expand the stabilizer in a transverse direction (e.g., by outward bowing as shown in FIG. 2). For example, the outer tube may be coupled-directly or indirectly—to a control element (e.g., actuation button 122 in the housing 102) such that a user may slide, rotate, depress, or otherwise manipulate the actuation button 122 to cause movement of the outer tube 105 (via any suitable linkage—not shown here) distally to push against the proximal end of stabilizer 112. While the control element is shown as an actuation button 122, the disclosure is not so limiting, and other types of control elements are within the scope of this disclosure. Similarly, proximal movement of the outer tube 105 may be effectuated by the actuator 122 (e.g., by rotating or sliding in an opposite direction) in order to collapse or undeploy the stabilizer.

Optionally, movement of the outer tube distally may cause automatic retraction of the sheath 114 proximally. For example, while not shown here, distal movement of the outer tube may cause release of a resilient member (e.g., a spring) from a compressed state that causes proximal retraction of the sheath (where the sheath is configured to be held in an initial state over the distal end of the elongate member via the compressed resilient spring.

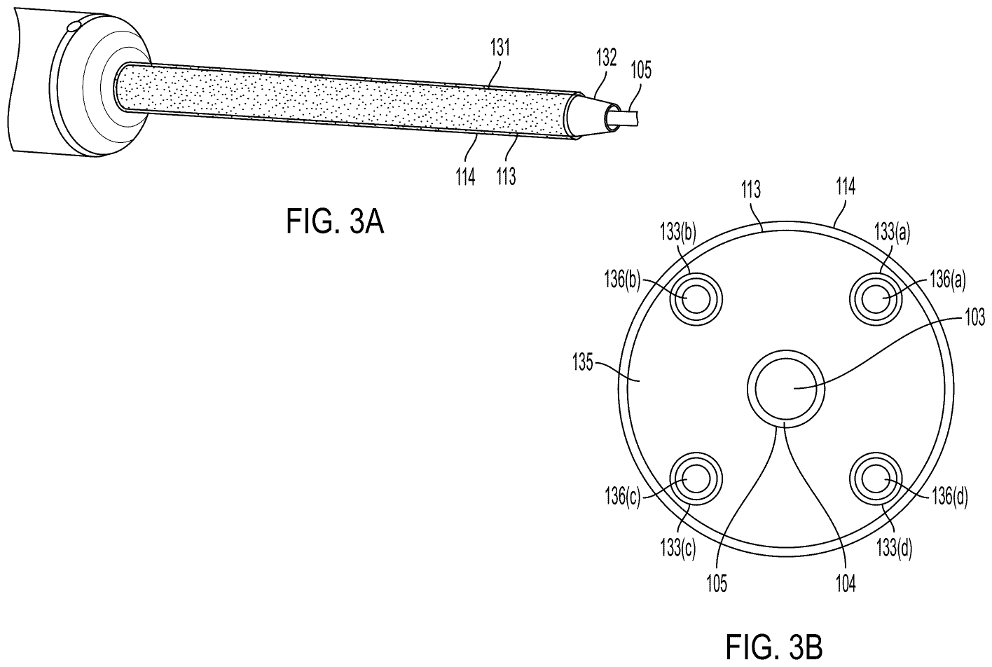

Referring back to FIGS. 1A-1E and FIG. 3A, the elongate member 113 comprises a proximal tubular portion 131 and a distal conical portion 132. The elongate member 113 defines a passageway or lumen 135 for receiving, without limitation, the guidewire lumen, the outer tube, the blood return channel, and a plurality of delivery tubes 133 (a)-(d) (collectively, 133) configured to deploy suture anchors. It should be noted that the passageway 135 and the outer tube 105 extends within the housing 102. FIG. 3B illustrates a cross-sectional view (along the y-z plane) of the passageway 135. Any number of delivery tubes may be included without deviating from the principles of this disclosure. It should be noted that while the disclosure describes the proximal tubular portion 131 as having a cylindrical profile, other profiles or shapes (such as square, triangular, pentagon, etc.) are within the scope of this disclosure.

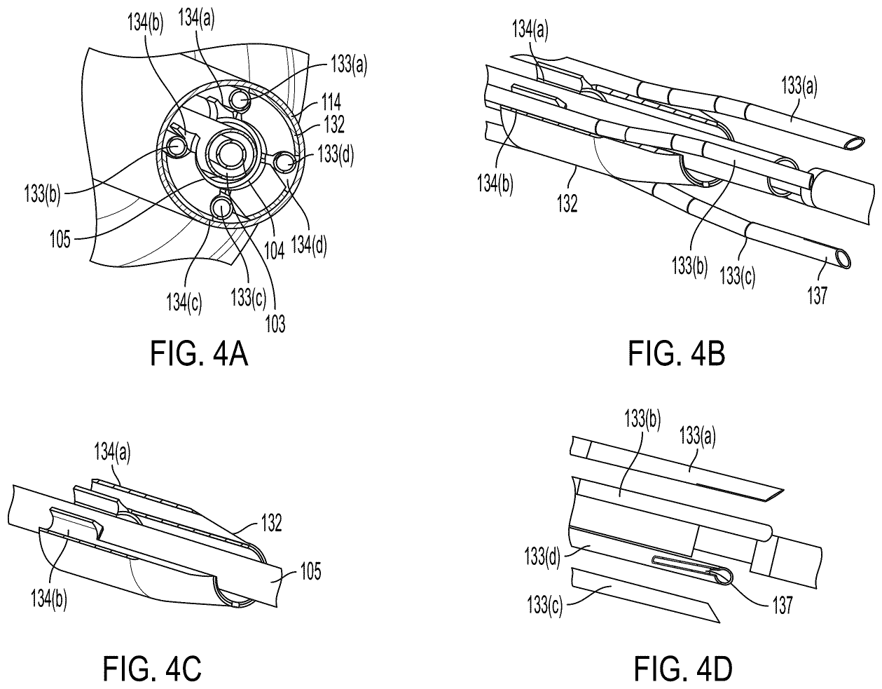

In various embodiments, the distal conical portion 132 is configured to provide a tapered distal end or tip to the elongate member 113 for ease of insertion within the target tissue as the delivery device is distally. As shown in FIGS. 4A-4C, the conical portion 132 may be configured to include one or more guiding elements or features 134 (a)-(d) (e.g., semi-circular slots shown in the figures) for receiving the delivery tubes in a desired configuration with respect to the guidewire lumen 103. Other types of guiding features such as, without limitation, channels, ramps, or the like are within the scope of this disclosure. In certain embodiments, the conical portion 132 may also include interfacing elements (e.g., slots) for removable attachment of suture systems such as suture tensioning devices, closure devices, etc. or components thereof (e.g., suture locks) which may be disposed within the passageway 135.

Each of the delivery tubes 133 (a)-(d) is a rigid, elongated tubular member that extends in the passageway 135, from a delivery tube holder 124 within the housing 102, distally towards the conical portion 132. Each delivery tube defines a lumen extending between its proximal and distal ends. The delivery tubes may have a circular cross-section, a square cross-section, or the like.

In a neutral position, the distal ends of the delivery tubes 133 (a)-(d) are configured to be situated at least a first threshold distance from the stabilizer and stored within the conical portion 132 in a desired configuration around the guidewire lumen (e.g., using the guiding features 134(a)-(d)). For example, the delivery tubes 133 (a)-(d) may be configured to radially surround the guidewire lumen 103 at a desired spacing. The first threshold distance may be determined to allow for easy insertion of the delivery tubes within the target tissue, for uninterrupted deployment of the stabilizer, and/or for uninterrupted confirmation of blood return.

The configuration of the delivery tubes is designed based on the desired configuration of the suture anchors upon deployment with respect to the guidewire and/or opening. Optionally, where the size of the delivery device needs to be minimized, the delivery tubes are arranged in a collapsed state around the guidewire lumen and then are expanded into the desired configuration when advanced out of the conical portion 132. For example, in some embodiments, one or more of the delivery tubes may each include a distal portion that flares out (or is bent outwards) with respect to the longitudinal axis of the tubular body of the delivery tube (shown in FIG. 4B) but stored in a collapsed state within the elongate member 102. FIG. 2B illustrates two of the delivery tubes having a distal portion that flares out (or is bent outwards) with respect to the longitudinal axis of the tubular body of the delivery tube while the other two delivery tubes have a straight axial profile (such that locations of the sutures when deployed form a rectangular cross-section). When such bent delivery tubes are advanced outside the conical portion 132, the bent distal portions are expanded to allow for suitable spacing of the delivery tubes and hence the suture anchors at with respect to a guidewire and/or an opening. In some other examples, the guiding elements 134 (a)-(d) may be configured to cause the delivery tubes achieve their desired configurations. For example, a guiding feature may be a small ramp to nudge the anchor delivery tubes outwards at a suitable distance from the guidewire lumen.

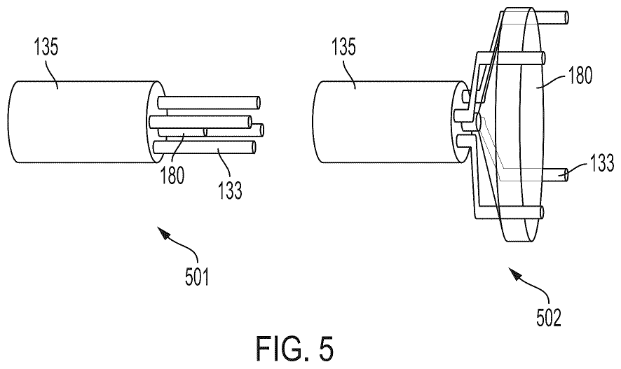

In yet another example, the delivery tubes are brought into their target configuration by advancing a positioning elements 180 within the elongate member 102 (shown in FIG. 5). The anchor delivery tubes in this example may be pre-bent or cut with laser into a pattern that allows them to be bent into a certain shape by the positioning element. In such embodiments, a positioning element 180 may be advanced within the passageway 135, between a space defined by the delivery tubes, to cause the delivery tubes 133 (a)-(d) to bend or flex outwards from an initial state 501 to a bent state 502 when the positioning element 180 is expanded (as shown in FIG. 5). The positioning element may be separate from or integral with any part of the device 100. They may be removably or fixedly mounted on the guidewire, the device and/or its components) and/or another device. Expansion of positioning element 180 may be selectively controlled to control positioning of the delivery tubes 133 (a)-(d) (and hence control the position of the sutures) with respect to the opening before deployment of the suture anchors without increasing the footprint of the delivery device itself. The positioning element may be, for example, an inflatable balloon, a disk, a cage, a ball, a mesh, a stent or other structure that is in a first compressed state for advancement within the passageway to a space between the delivery tubes. and may assume a second expanded state to bend the delivery tubes outwards.

After stabilizer deployment, the delivery tubes may be advanced distally out the distal end of the conical portion 132 (in a desired configuration as discussed above) and towards the stabilizer until they the distal ends of the delivery tubes 133 (a)-(d) are a second threshold distance from the deployed stabilizer. The second threshold distance may be determined to allow the stabilizer to control accurate positioning of the delivery tubes for suture deployment with respect to an opening. Optionally, in order to ensure that the anchors are deployed in the target tissue, the delivery tubes may be advanced a suitable distance past the stabilizer which is on the other side of the target tissue. Since the anchor delivery tubes may travel through soft tissue, e.g. subcutaneous tissue for deploying the anchored sutures on a vessel wall, the distal ends of the delivery tubes may have a profile (e.g., beveled edge, shape edge) to facilitate passage of the delivery tubes through the soft tissue.

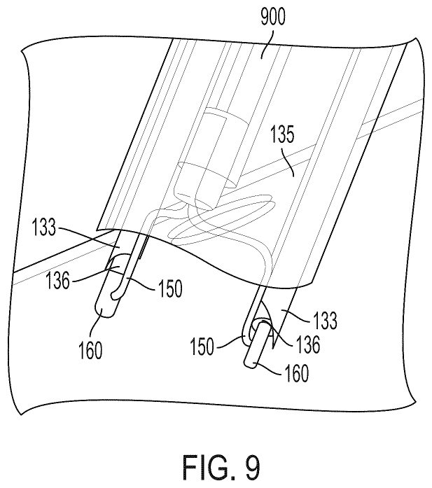

Optionally, as shown in FIGS. 4B and 4D each of the delivery tubes 133 (a)-(d) includes a longitudinal slot 137 extending from the corresponding distal end along the longitudinal length of that delivery tube (the slot may not extend along an entire length of a delivery tube). Optionally, the slot 137 may be an opening or a hole formed in a side of a delivery tube that does not extend to a distal end of the delivery tube. Optionally, the slot may be provided in the portion of the delivery tube that faces the guidewire lumen (e.g., when a tensioning tube is stored within the conical portion 132). However, the slot may be provided in any portion of the delivery tube (e.g., away from the guidewire lumen). As shown in FIG. 9, in various embodiments, the distal ends of the sutures 150 may be coupled to suture anchors 160 and stored within the distal ends of the delivery tubes 133 (a)-(d) while the proximal ends of the sutures 150 exit the delivery tubes via the corresponding slots (one suture per delivery tube) to be engage with a suturing system 900 (e.g., a closure device, a tensioning tube, or the like). The distal ends of the sutures 150 coupled to the suture anchors 160 are deployable via the distal ends of the delivery tubes 133 (a)-(d).

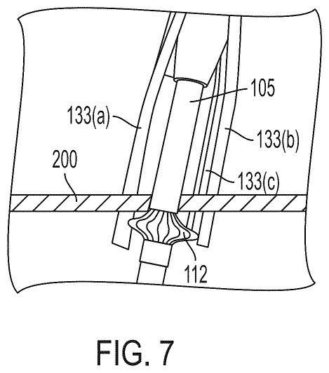

The delivery tubes 133 (a)-(d) may be advanced within the passageway 135 to exit out the conical portion 132 such that the distal end of each delivery tube may be positioned proximate or within a tissue region (e.g., within a vessel wall) and/or structure surrounding an opening to be closed for subsequent deployment, desired positioning, and anchorage of the distal ends of the sutures around a periphery of the opening (as discussed below). In various embodiments, the lengths of the delivery tubes may be configured such that their respective distal ends reach the target tissue simultaneously. As such, if the delivery device is advanced at an angle (that is not) 90° with respect to the target tissue, the lengths of the delivery tubes may vary with respect to each other to ensure that each of them reach the target tissue simultaneously. For example, FIG. 7 illustrates unequal length delivery tubes 133 (a)-(d) such that they reach and/or are inserted within the target tissue 200 at the same time.

In some embodiments, delivery tubes vary in size depending on the size of the suture anchor being used. In some examples, a delivery tube has an outer diameter (OD) of about 0.030″, about 0.032″ about 0.033″, about 0.034″, about 0.035″ about 0.037″, about 0.040″, or the like; and an inner diameter (ID) of about 0.023″, about 0.025″, about 0.026″, about 0.027″, about 0.029″, about 0.030″, or the like. In some other examples, the anchor delivery tubes have an OD of about 0.030″−0.040″, about 0.032″-0.038″, about 0.034″-0.036″, or the like; and an ID of about 0.020″−0.030″, about 0.022″-0.028″, about 0.024″-0.026″, or the like. The inner and outer diameter of the delivery tubes can vary depending on the size of the suture anchor disposed within the delivery tube, size/diameter of the outer tube, diameter of the passageway, size of the opening, or the like.

Further, the disclosure illustrates four delivery tubes 133 (a)-(d), but this is merely for illustrative purposes. The precise number and spacing of delivery tubes can be left to the particular application. It is anticipated that the larger an opening the larger the number of sutures and hence a larger number of delivery tubes are required. In some applications, the number of sutures may also depend on, for example, force needed to pull together tissue surrounding an opening, a location of the opening, a type of the opening, a shape of the opening, or the like. Regarding the spacing, the delivery tube may be evenly (e.g., radially around a guidewire lumen) and/or unevenly spaced within the passageway 135.

Various forms of the suture anchors are described below in more detail. In various embodiments, when a suture anchor 160 resides within the corresponding delivery tube 133, it is configured to be in a first compressed form for movement within the delivery tube lumen and for insertion within the tissue. Once inserted or deployed within the tissue, the suture anchor 160 is configured to open or expand, and anchor the distal end of the corresponding sutures to the tissue.

The suture anchors 160 can be made of super elastic or shape memory nitinol material wire(s). The nitinol wire(s) may be super-elastic and permit the anchors to assume a compressed state for insertion through the vascular wall without catching on or damaging the vascular wall. For example, the nitinol material wire(s) may be preformed by heat treating into a curvature, for example a hook shape, that will act as a suture anchor. The curvature in the wire(s) can be straightened out by drawing into the delivery tube 133. After the suture(s) is advanced out of the delivery tube(s) and the curvature reforms to anchor the suture. Similarly, nitinol tubes may be laser cut into a hook shape and shape-set by heating the cut tube to a certain temperature. Then, the nitinol tubes can be pulled into their original tube shape within the tube, then return to their curved hook shape when removed from the tube upon deployment. Optionally, the anchors may be disposed within removable sheaths that may be removed allowing the anchors to return to their hook shape.

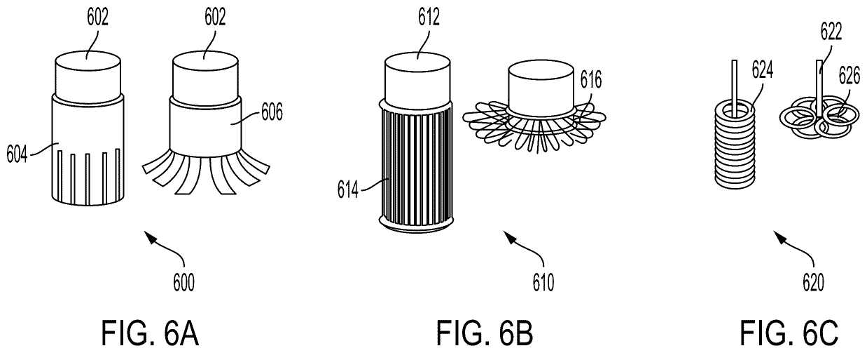

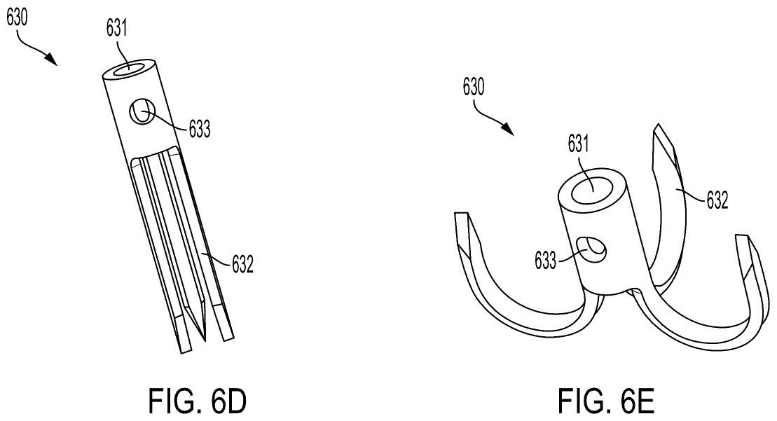

Referring to FIG. 6A-6E, various types of suture anchors are possible, such as tube-based nitinol anchors 600, wire-based nitinol anchors 610 and suture knot anchors 620. Tube-based anchor 600 is illustrated at the end of suture 602. In a compressed or not expanded form 604, the anchor may be about the same diameter or slightly larger than the suture 602. When the anchor 600 is no longer compressed (e.g., within a delivery tube) and is permitted to reach its expanded shape 606 (e.g., when pushed out of a delivery tube), the anchor may have a plurality of hooks or fingers that flare out and get embedded within one or more layers of tissue proximate the opening to be closed.

Similarly, wire-based anchor 610 may have a compressed form 612 (e.g., when it is inside a delivery tube) and in an expanded form 616, have a variety of small wires that spring out embed within the tissue. Suture based anchors 620 (see FIG. 6C) may include a cylindrical knot of suture 624 that is inserted into the tissue. When the length of suture 622 is pulled on to the tension the suture 622, the knot 624 can be pulled into an expanded form 626, which can attach to the tissue. Another example embodiment of a tube-based anchor is illustrated by FIGS. 6D and 6E, which show a three-prong laser cut tube-based anchor. As described above, a nitinol tube may be laser cut into a hook shape and shape-set by heating the cut tube to a certain temperature. Then, the tubes can be pulled into their original tube shape into the delivery tubes of device 100, then return to their curved hook shape when removed from the delivery tubes. Tube-based anchor 630 can include a tube body with a bore 631 running therethrough. Prongs 632 and/or hole 633 may be laser cut into the tube. Hole 633 can be used to attach a suture to tube-based anchor 630, as described above. For example, a knot or reflowed suture ball may prevent a suture run through 631 from pulling back through hole 633. After tube-based anchor 630 is cut, it may heat formed into the hook shape illustrated in FIG. 6E. For example, prongs 630 may be formed into hooks that can engage with tissue and attach to one or more layers of the tissue surface. Tube-based anchors may be compressed (e.g., a shape such as that illustrated by FIG. 6D) inside a deployment device such as a tensioning tube and/or a delivery tube of the current disclosure. When released from the deployment device, tube-based anchor 630 can return to deployed hook shape illustrated in FIG. 6E. While the anchors of FIG. 6 are described as either nitinol or suture-based anchors, anchors may be made from other suitable materials. Such anchors can provide other advantages in addition to ease of anchorage of sutures and relative strength. Though not illustrated in FIG. 6, other types of anchors are also possible, such as pledget-based anchors. In some examples, one or more of the anchors may have additional features to facilitate radial cinching of an opening. For example, a hole (or any other opening) may be provided on a side of an anchor (e.g., 633 in the side of tube based anchor shown in FIGS. 6D and 6E) for suture exit, where a suture exiting from a side (e.g., radially) when tensioned will create a radial or sideway force instead of an outward pulling force.

While the figures illustrate anchor-based sutures, the disclosure is not so limiting and non-anchor-based sutures may similarly be used without deviating from the principles of this disclosure. For example, sutures can be tied with one another and form a knot by going over themselves within tissue to close the opening. In other examples, a suture knot in low profile could be delivered within the tissue and then pulled to form a larger knot that can act as an anchor.

Referring back to FIGS. 1A-1E, a pusher rod (136 (a)-(d)) (or push rod) that movably extends distally from a pusher rod holder 126 is axially disposed within the lumen of each of the delivery tubes (i.e., each pusher rod extends distally inside the delivery tubes from the proximal ends of the corresponding delivery tubes). In various embodiments, distal ends of each of the push rods 136 (a)-(d) are configured to push the corresponding suture anchors 160 out the distal ends of the delivery tubes 133 (a)-(d) when the delivery tubes are suitable positioned. In various embodiments, initially the pusher rods 136 and the delivery tubes 133 are distally advanced synchronously. However, when the delivery tubes have reached their maximum allowed advancement position, the delivery tubes stop advancing forward while the push rods continue to advance so that they push the anchors 160 out of the distal ends of the delivery tubes. The maximum allowed advancement position of the delivery tubes is determined relative to the deployed stabilizer inside the target tissue (e.g., the vessel) to ensure anchors are deployed on the target tissue wall (e.g., vessel wall) and not in the fascia outside the target tissue. The maximum allowed advancement position delivery tubes may be controlled by constraining the advancement of the delivery tube holder 124 using a resilient member as discussed below.

Upon deployment of the anchors, the delivery tubes and the push rods retract automatically to avoid putting continuous pressure on the target tissue. Optionally, the delivery tubes and the push rods may be automatically partially retracted if they encounter stiff resistance to advancement in order to prevent increase in deployment forces when resistance in deployment is experienced (for example from severe calcification).

The advancement and retraction of the delivery tubes and the push rods is controlled by suitable mechanisms disposed within the housing 102. As discussed above, the housing 102 includes control elements and mechanisms that may be manipulated by a user to advance and trigger various functions of the delivery device such as, without limitation, retraction of the sheath, deployment of the stabilizer, advancement of the delivery tubes, advancement of the push rods for deployment of the anchored sutures, retraction of the delivery tubes and push rods, retraction of the stabilizer, or the like. Optionally, the housing is configured to have a shape (e.g., the shape of a handle) and/or positioning of the control elements such that a user can easily hold the housing and manipulate the control elements using one and/or two hands. Examples of such mechanisms or control elements may include, without limitation, a slider or plunger coupled to a linear advancement type mechanism; a push button coupled to a spring or resilient member type mechanism; a rotational knob-based mechanism, a flip handle coupled to a rack and pinion type mechanism; a squeeze handle type mechanism; or the like.

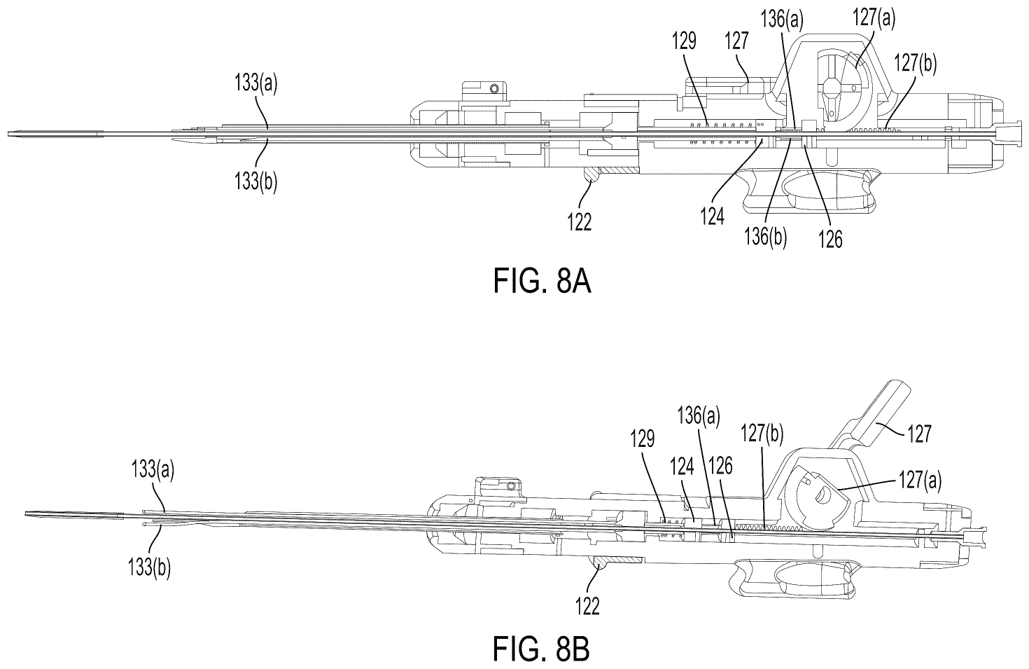

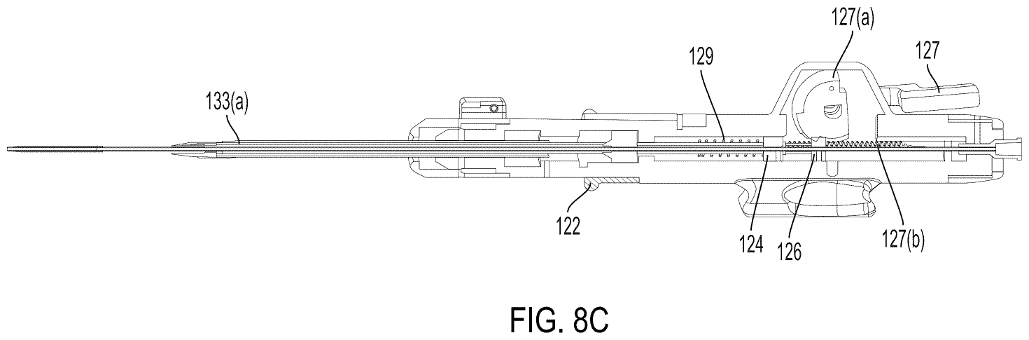

For example, FIGS. 8A-8C illustrate a flip handle coupled to a rack and pinion type mechanism. As shown in the figures, the pinion 127 (a) is coupled to the flip handle 128 such that the rack and pinion mechanism is actuated by a flip handle 128. The rack 127 (b) is coupled to the delivery tube holder 124 and the push rod holder 126. As such, when the flip handle 128 is rotated, it rotates the pinion 127 (a), the rotation being translated to linear movement of the rack 127 (b) to suitably move and/or position the delivery tube holder 124 and the push rod holder 126. Movement of the delivery tube holder 124 and the push rod holder 126 in the distal direction causes advancement of the delivery tubes and the push rods, and movement of the delivery tube holder 124 and the push rod holder 126 in the proximal direction causes retraction of the delivery tubes and the push rods.

Further as shown in the figures, a resilient member 129 (e.g., a spring) is configured to restrain the distal movement of the delivery tube holder 124. Specifically, as the delivery tube holder 124 moves distally (via the movement of the rack 127 (b), it compresses the resilient member 129. The maximum advancement position of the delivery tubes is, therefore, governed by the allowed maximum compression of the resilient member 129. Once the resilient member 129 is in its maximum compressed state, it prevents any further distal advancement of the delivery tube holder 124 (while the push rod holder can still continue to be distally advanced to deploy the suture anchors by pushing them out of the delivery tubes). After deployment of the sutures, continued rotation of the flip handle 128 in the proximal direction retracts the delivery tube holder 124 and the push rod holder 126 back into the housing 102 (the resilient member 129 is returned to its resting state).

FIGS. 8A-8C illustrate cross section views of the housing 102 in a neutral position, in a deployment position, and in a retracted position, respectively. As shown in FIG. 8A, in the neutral position, the flip handle 128 is positioned to face the distal end of the delivery device, and the delivery tubes are stored within the conical section of the elongate member. As the flip handle is rotated towards the proximal end of the device, the delivery tubes and the push rods are advanced distally until the sutures are deployed (FIG. 8B). Continuous rotation of the flip handle causes retraction of the delivery tubes and the push rods after deployment of the anchors (FIG. 8C).

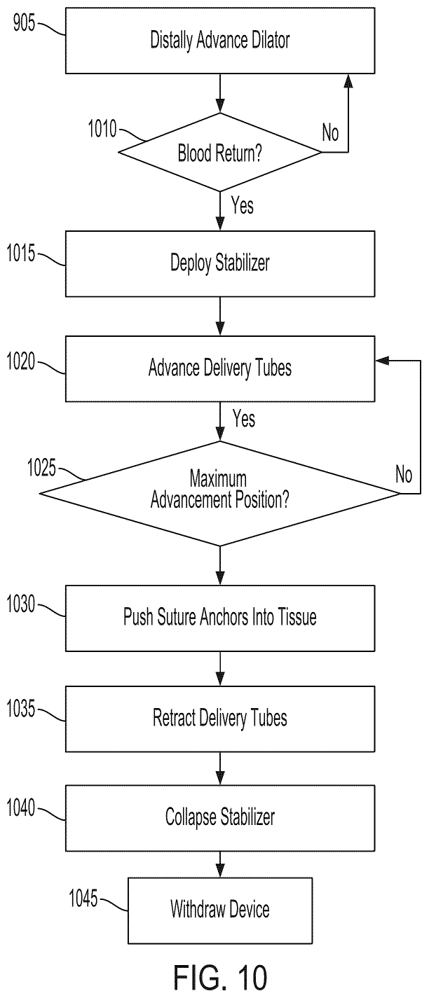

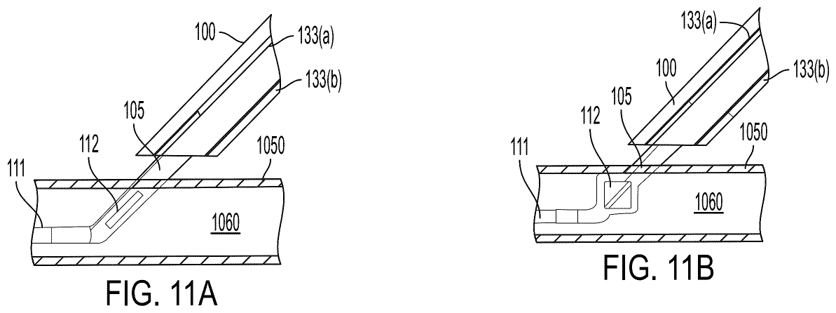

FIG. 10 is a flowchart depicting an example method 1000 for using a delivery device 100 of this disclosure. At step 1005, the dilator may be distally advanced into the target tissue (e.g., a vessel wall) by distally advancing the delivery device over a guidewire. Once blood return is visually detected via the proximal port of the blood return channel (1010=YES), the stabilizer may be deployed at 1015.

Next, at 1020, the delivery tubes (including the push rods) may be advanced into the target tissue until the delivery tubes reach their maximum advancement position. Once the delivery tubes reach their maximum advancement position (1025=YES), the push rods may push the suture anchors into the target tissue for deployment (1030). The exact location which the sutures and suture anchors deployed about a periphery of the opening can vary depending on the size, shape, and location of the opening.

Once the sutures are deployed, the delivery tubes including the push rods are retracted back into the delivery device (1035). Finally, the stabilizer may by undeployed by collapsing (1040), and the delivery device is withdrawn (1045).

As the delivery device is withdrawn, proximal ends of the anchored sutures may be manipulated using, for example, a tensioning device of a closure system to cinch the opening.

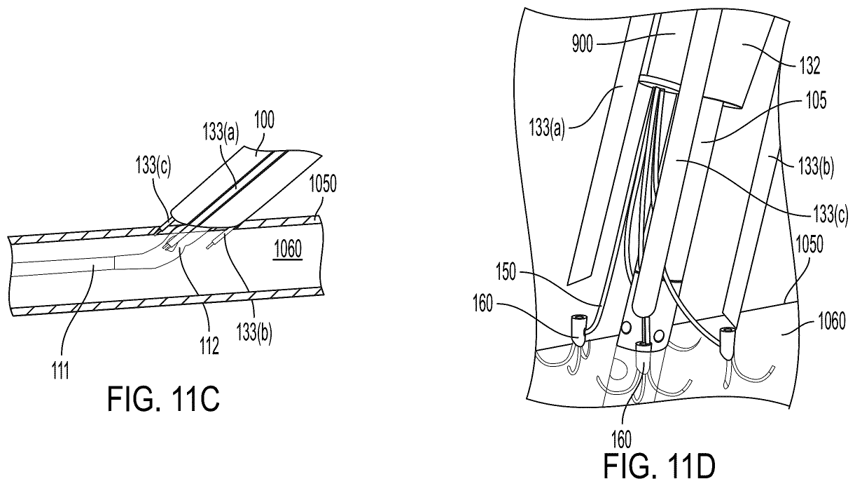

FIG. 11 provides a visualization of the above process of using the delivery device 110 to deploy sutures proximate to an access site in a vessel lumen. The example in FIG. 11 shows four anchor-based sutures deployed around an access site 1150 in a vessel wall 1160. For example, FIG. 11A illustrates the dilator being advanced into the vessel lumen 1170, while the stabilizer is in a collapsed state. The distal portion of the device is advanced within the lumen 1170 until blood return is observed. FIG. 11B illustrates the stabilizer in a deployed state for proper positioning of the delivery tubes when deployed. FIG. 11C illustrates the delivery tubes deployed within the vessel lumen and positioned at their maximum advancement position. In this position, distal advancement of the push rods causes deployment of the suture anchors by pushing them out the distal ends of the delivery tubes (suture anchors are partially visible in FIG. 11C). FIG. 11D illustrates suture anchors in their deployed state and the delivery tubes being retracted, while proximal ends of the sutures are manipulated by a suitable closure system (900).

Each of the plurality of sutures may be individually deployed. In various embodiments, the arrangement of suture (once deployed around a vascular opening) may be configured to enable radial cinching by pulling the sutures (example, via suture anchors) towards a center of the suture arrangement where the vascular opening is located. For example, the suture may be deployed into tissue and/or structures around the opening such that when the sutures are tensioned the sutures radially cinch the opening inwards by pulling on the tissue and/or structures. Similarly, the sutures may be deployed in a manner that they form approximate vertices of a square, triangle, pentagon, hexagon, or the like with the opening being between the arrangement (center or off center). Additionally, sutures may be deployed asymmetrically about a vascular access site or opening depending on the size and shape of the access site or opening. Other arrangements are within the scope of this disclosure such as to allow for linear cinching, or the like. The deployed sutures may then be cinched, for example, using the closure systems discussed above (e.g., whose described in for example, U.S. Pat. No. 11,382,609, U.S. Patent Application Publication No. 20220175356, U.S. Patent Application Publication No. 20230149005, and U.S. Patent Application Publication No. 20230309979). For example, dynamic cinching of sutures may be achieved using a resilient member (e.g., a spring) where sutures pass through a tubular element and are coupled to the resilient member. Specifically, cinching of the sutures may be turned on and/or off, precisely modulated, and/or clocked at a desired cinching force upon completion of a procedure (such that access is no longer needed) by manipulation of the resilient member.

The above disclosure describes a suture delivery device for deployment of sutures first followed by advancement of an interventional device such as a large bore sheath or catheter (e.g., over a guidewire) into the access site. However, often dynamic cinching of an access site may be desired after an interventional device has already been inserted within the access site such that the diameter of the access site has already been enlarged (referred to as a post-procedure closure). An “enlarged access site”, as used herein, refers to an access site that has a diameter of about 8-30 French for providing arterial access or about 12-40 French for providing venous access, via an interventional device. Deployment of sutures around a larger diameter access site for, for example, homeostasis and/or reversible closure of the access site upon removal of an interventional device can present certain practical problems. For example, removal of an interventional device from an access site will cause blood to flow out of the access site and, therefore, presence of blood in the blood return channel of the example device discussed above cannot be used as a reliable indication of correct positioning of the stabilizer (and, hence, the correct depth of the distal tip of the delivery device being inserted) within the vessel lumen. Moreover, given the already enlarged diameter of the access site, the stabilizer may not be able to provide reliable fixation of the delivery device during deployment of the sutures (for e.g., the stabilizer may be pulled out of the enlarged access site). Finally, when the access site diameter is enlarged, the suture anchors may be deployed into the cut portion of the vessel wall surrounding the access site instead of the vessel wall itself creating unstable and/or inaccurate suture deployment which will lead to incomplete cinching of the access site.

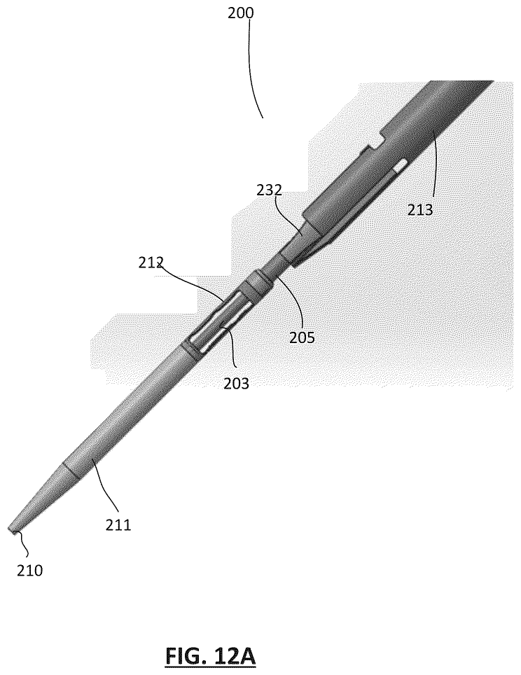

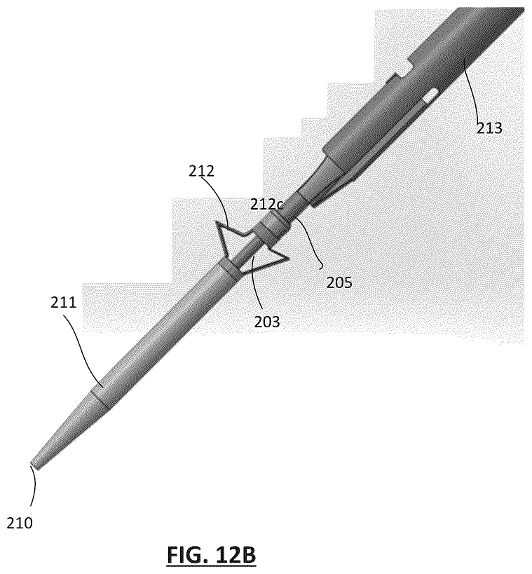

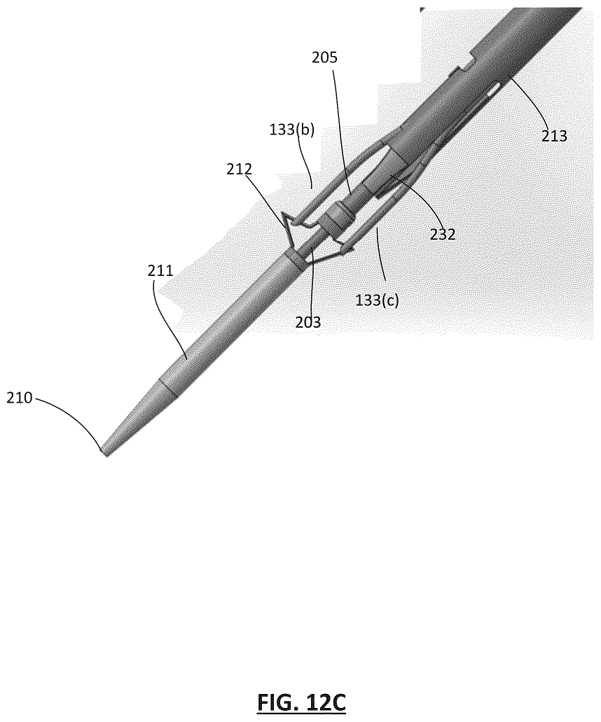

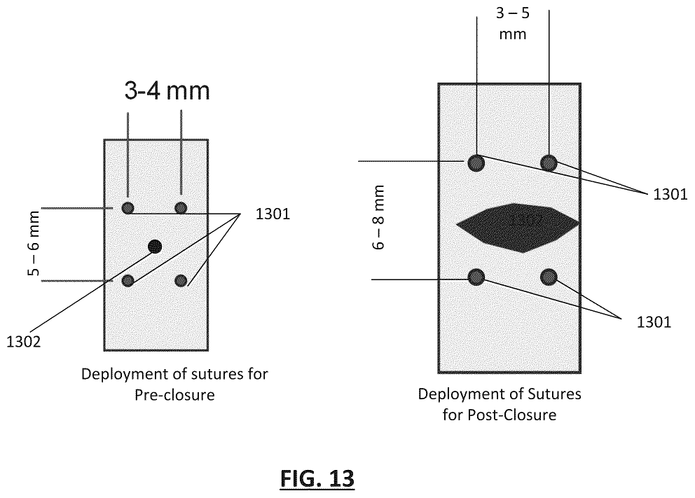

An example delivery device for deployment of sutures for a post-procedure closure of an already enlarged access site is described with respect to FIGS. 12A, 12B, and 13. To facilitate description, in the following embodiments of the disclosure, the same or similar reference numerals are used to indicate the same or similar elements, and the description of the same technical contents is omitted. For the description of the omitted parts, reference may be made to the foregoing embodiments, which are not repeated herein.

FIG. 12A illustrates a suture delivery device 200 with a stabilizer 212 in a collapsed state while FIG. 12B illustrates the suture delivery device 200 with the stabilizer 212 in an expanded state. In the embodiments shown in FIGS. 12A and 12B, the diameter of the proximal portion (211a) of the dilator 211 is about 10-20 French (about 0.13-0.26 inches), about 10-15 French, about 12-18 French, about 14-16 French, about 12-16 French, or other suitable diameters. The increased diameter of the dilator 111 allows for plugging of the access site with the dilator in order to prevent blood loss through the access site upon removal of the interventional device.

Optionally, the length of the dilator 111 may also be increased to about 1.5 to about 2 inches, about 1.6 inches to about 1.9 inches, about 1.7 inches to about 1.8 inches, about 1 inch to about 5 inches, about 2 inches to about 4 inches, or the like. Increasing the length of the dilator 111 allows for stopping leakage of blood completely from the access site upon insertion of a suitable length of the dilator 111 (close to 80-100% length of the dilator) within the access site before the distal port 241 (a) of the blood return channel 204 reaches is positioned within the vessel to cause flow of blood to the proximal port 241 (b) the blood return channel 204. In the absence of blood leakage from the access site allows for precise observation of feedback relating to accurate positioning of the stabilizer within the vessel (i.e., target depth of the dilator being inserted within the vessel lumen) when the distal port 241 (a) reaches inside the vessel and gives indication with a pulsatile blood flow from the proximal port 241 (b).

In certain embodiments, the footprint of the stabilizer 212 is also increased so as to ensure accurate referencing of the anterior vessel wall with respect to the access site. In some embodiments, the wing span of each of the wings 212a and 212b may be about 1-3 mm, about 1.5-2.5 mm, about 2 mm, etc. so as to provide adequate anchorage despite the enlarged diameter of the access site. Optionally, the stabilizer is deployed in the longitudinal direction (i.e., along the vessel wall or along the long axis of the vessel) to provide maximum anchorage. The stabilizer is coupled to the outer tube 205 such that the stabilizer 112 may be deployed by movement of the outer tube 205 distally such that the distal end of the elongate member pushes against the proximal end of stabilizer 212 to shorten or compress the axial length of the stabilizer and expand the stabilizer in a transverse direction (e.g., by outward bowing as shown in FIG. 12A and FIG. 12B). For example, a coupling element 212 (c) may be provided that is configured to coupled to the proximal end of the stabilizer and also receive the distal end of the outer tube such that movement of the outer tube distally causes distal movement of the coupling element 121 (c) to cause compression of the stabilizer (or vice versa).

In some embodiments, the delivery tubes 233 (a)-(d) may also be configured for deployment of suture anchors around a larger access site. Specifically, the target locations for deployment of the sutures may be optimized to cover a larger access site. For example, for deployment of sutures around a small access site, the suture deployment locations may form a small cross sectional area of about 3-5 mm (longitudinal length) times about 5-6 mm (axial length). The profile of the area between the deployed suture locations may be square when delivery tubes have a straight axial profile, rectangular when two delivery tubes have an axial offset at or proximate their distal portions (as shown in, for example, FIG. 2B) when seen in a top view, and/or another shape (e.g., pentagonal when there five delivery tubes). For deployment of sutures around a larger access site, the cross section of the area between the deployed suture locations may be increased by, for example, providing delivery tubes that all (i.e., all four delivery tubes) have an axial offset at or proximate their distal portions (i.e., the distal portions flares out or is bent outwards) as shown in FIG. 12C, and/or increasing the axial offset of one or more of the delivery tubes. The larger cross section may be about 3-5 mm (longitudinal length) times about 6-8 mm (axial length). FIG. 13 a top view of the locations of the deployed sutures (1301) and the corresponding cross-sections formed using the pre-closure delivery device (FIG. 1) and the post-closure delivery device (FIG. 12A), respectively, around an access site 1302.

Additionally and/or alternatively, the cross section of the stabilizer may be expanded using a positioning element, as discussed above with respect to FIG. 5.

It should be noted that the methods for using the delivery device for post-closure of the an enlarged access site will be the same as that described with respect to FIG. 10 with the delivery device being advanced within an already enlarged access site.

Kits for closure of openings in a target tissue are also provided. In general, the kits comprise a suture delivery device including sutures and suture anchors, and one or more other components of the delivery device (that can be removably attached). The distal ends of the sutures coupled to suture anchors may be disposed within the delivery tubes to be stored within a distal end of each delivery tube. The proximal ends of the sutures may exit the delivery tubes (e.g., via the slots discussed above) to be manipulated using any now or hereafter known methods and devices. The kit can also include a suture closure system (e.g., including a tensioning device).

An interventional device for accessing the opening may be included in the kits. Optionally, a guidewire may be included in the kits.

In addition, as evidenced by the description of the devices and methods above, it may be advantageous to provide a kit with additional tools useful in carrying out the described methods. For example, the kits may further comprise a cutting wire for cutting the sutures, a stabilizer, a positioning element, and/or a suture lock.

The kit may also include instructions on how to use the contents of the kit. For example, Optionally, the kits may include instructions for deploying the sutures using the delivery device. Instructions may include reference materials (including indications for use, etc.) and be in any appropriate format, including written, pictographic, visual, electronic, etc., and be in any language, or multiple languages.

It will be understood that terms such as “same,” “equal,” “planar,” or “coplanar,” as used herein when referring to orientation, layout, location, shapes, sizes, amounts, or other measures do not necessarily mean an exactly identical orientation, layout, location, shape, size, amount, or other measure, but are intended to encompass nearly identical orientation, layout, location, shapes, sizes, amounts, or other measures within acceptable variations that may occur, for example, due to manufacturing processes. The term “substantially” may be used herein to emphasize this meaning, unless the context or other statements clearly indicate otherwise. For example, items described as “substantially the same,” “substantially equal,” or “substantially planar,” may be exactly the same, equal, or planar, or may be the same, equal, or planar within acceptable variations that may occur, for example, due to manufacturing processes and/or tolerances. The term “substantially” may be used to encompass this meaning, especially when such variations do not materially alter functionality.

It will be understood that various modifications may be made to the embodiments disclosed herein. Likewise, the above disclosed methods may be performed according to an alternate sequence. Therefore, the above description should not be construed as limiting, but merely as exemplification of the various embodiments. Those skilled in the art will envision other modifications within the scope and spirit of the claims appended hereto.

What is claimed is:

1. A delivery device for suture deployment for post-procedure closure of an access sire, the delivery device comprising:
a distal assembly comprising an elongate member and a dilator, the dilator having a diameter of about 10-20 French;
a proximal housing comprising one or more control elements for controlling one or more components of the delivery device; and
a plurality of delivery tubes extending distally from the proximal housing into the elongate member, each of the plurality of delivery tubes comprising:
a suture anchor stored within a distal portion of that delivery tube, and
a push rod configured to push the suture anchor distally out of the distal portion of that delivery tube for deployment of the suture anchor within a target tissue around the access site after removal of an interventional device from the access site.

2. The delivery device of claim 1, further comprising a guidewire lumen extending between a distal end and a proximal end of the delivery device.

3. The delivery device of claim 1, further comprising a blood return channel extending between a distal port disposed within the distal assembly and a proximal port disposed within the proximal housing, the blood return channel being configured to provide an indication of proper positioning of the distal assembly within the target tissue based on presence of blood in the proximal port.

4. The delivery device of claim 3, wherein the blood return channel is a passageway defined by an inner guidewire lumen and an outer tube.

5. The delivery device of claim 3, wherein a length of the dilator is configured to plug the access site to prevent leakage of blood from the access site such that presence of blood in the proximal port provides the indication of proper positioning of the distal assembly within the target tissue.

6. The delivery device of claim 3, wherein the length of the dilator is about 1-5 inches.

7. The delivery device of claim 1, wherein the distal assembly further comprises a stabilizer, the stabilizer having a total wing span of about 2 mm to about 6 mm in an expanded state.

8. The delivery device of claim 7, wherein the stabilizer is disposed between the dilator and the elongate member and has a diameter that is substantially similar to that of the dilator in an undeployed state.

9. The delivery device of claim 8, wherein distal movement of an outer tube within the elongate member causes deployment of the stabilizer for providing temporary fixation of the delivery device at a location in the target tissue that will correctly position the suture anchors upon deployment.

10. The delivery device of claim 1, further comprising a removable sheath mounted over at least a distal portion of the elongate member.

11. The delivery device of claim 1, wherein the elongate member comprises:
a proximal tubular portion; and
a distal conical portion comprising a plurality of guiding features configured to position the plurality of delivery tubes in a desired configuration upon deployment.

12. The delivery device of claim 1, further comprising a passageway extending from a distal end of the elongate member into the proximal housing, the passageway being configured to receive a guidewire lumen, the plurality of delivery tubes, and an outer tube configured to define a blood return channel.

13. The delivery device of claim 12, wherein each of the plurality of delivery tubes comprises an elongate tubular member extending distally, from a delivery tube holder in the proximal housing, into the passageway.

14. The delivery device of claim 13, that push rod extends distally into a lumen of that delivery tube from a push rod holder in the proximal housing.

15. The delivery device of claim 14, wherein the proximal housing comprises a rack and pinion control mechanism configured to be actuated by a flip handle such that rotational motion of the flip handle causes distal advancement of the delivery tube holder and the push rod holder.

16. The delivery device of claim 15, further comprising a resilient member configured to stop distal movement of the delivery tube holder when in a fully compressed state.

17. The delivery device of claim 16, wherein distal movement of the push rod holder upon stopping of distal movement of the delivery tube holder causes, via the push rod, deployment of the suture anchor within the target tissue.

18. The delivery device of claim 1, wherein each of the plurality of delivery tubes comprises a slot for receiving a distal end of a suture, the distal end of the suture being coupled with a suture anchor.

19. The delivery device of claim 18, further comprising a positioning element disposed within a passageway including a guidewire lumen and configured to cause flaring of the plurality of delivery tubes outwardly with respect to the guidewire lumen upon deployment.

19. The delivery device of claim 18, wherein each of the plurality of delivery tubes comprises a distal portion that flares outwardly with respect to the proximal portion of that delivery tube such that the sutures are deployed to form an area having a rectangular cross section of about 3 mm×8 mm.

20. A method of deploying sutures using the delivery device of claim 1, the method comprising:
advancing a distal end of the delivery device into a previously enlarged access site until the access site is plugged to prevent leakage of blood from the access site;
deploying, upon observation of blood return, a stabilizer;
distally advancing the plurality of delivery tubes into the target tissue; and
distally advancing, upon detecting that the plurality of delivery tubes have reached a maximum advancement position, each push rod into a lumen of that delivery tube to cause deployment of that suture anchor.
